(19) United States
(12) Patent Application Publication
(10) Pub. No.: US 2026/0061025 A1
(43) Pub. Date: Mar. 5, 2026

(54) Polyhomoarginine (PHA) Polymers And Uses Thereof

(71) Applicant: Singapore Health Services Pte Ltd, Singapore (SG)

(72) Inventors: Rajamani LAKSHMINARAYANAN, Singapore (SG); Jodhbir Singh MEHTA, Singapore (SG); Tun Aung THET, Singapore (SG)

(21) Appl. No.: 19/314,948

(22) Filed: Aug. 29, 2025

(30) Foreign Application Priority Data
Aug. 30, 2024 (SG) 10202402699R

(51) Int. Cl. A61K 38/08; A61K 9/00; A61K 31/4174; A61K 31/4178; A61K 31/4196; A61K 31/496; A61K 31/506; A61K 31/7048; A61P 27/02; A61P 31/04; A61P 31/10

(52) U.S. Cl. CPC A61K 38/08; A61K 9/0051; A61K 31/4174; A61K 31/4178; A61K 31/4196; A61K 31/496; A61K 31/506; A61K 31/7048; A61P 27/02; A61P 31/04; A61P 31/10

(57) ABSTRACT

The present invention relates to the use of polyhomoarginine comprising 6-26 homoarginine residues or a pharmaceutically acceptable salt thereof, in the treatment of an infection, wherein the infection is a fungal infection, a bacterial infection or a combination thereof. The present invention also relates to the use of polyhomoarginine comprising 6-26 homoarginine residues or a pharmaceutically acceptable salt thereof in a formulation or in a contact lens.

### TECHNICAL FIELD

This invention relates to the use of polyhomoarginine in the treatment of an infection. The present invention also relates to the use of polyhomoarginine in a formulation or in a contact lens.

### BACKGROUND ART

Infectious disease are disorders caused by microorganisms such as virus, fungi, bacteria, and parasites. Keratitis is a severe sight-threatening infectious disease, and in particular, fungal keratitis or mycotic keratitis affects millions of people world-wide, especially in tropical and subtropical countries such as Asia, Latin America, and Africa. Fungal keratitis remains a silent epidemic and the major cause of blindness and vision loss. The prognosis of fungal keratitis such as Fusarium keratitis is very poor, as it causes more debilitating effects on the eyes compared to other infectious keratitis. Natamycin is the only US Food and Drug Association (FDA) approved antifungal and was recently listed in WHO essential medicine for fungal keratitis, which is the best for the filamentous fungi, Fusarium. However, the global availability and affordability are still poor and the Natamycin experiences poor penetration to the corneal stroma, which is the usual site for fungal organisms to inhabit. Moreover, natamycin has low bioavailability.

In view of the above, there is a need for the development of a treatment for an infectious disease that overcomes or at least ameliorates, one or more of the disadvantages described above.

### SUMMARY

In an aspect, there is provided the use of polyhomoarginine (PHA) comprising 6-26 homoarginine residues or a pharmaceutically acceptable salt thereof, in the manufacture of a medicament for the treatment of an infection, wherein the infection is a fungal infection, a bacterial infection or a combination thereof.

Advantageously, a low molecular weight polyhomoarginine, such as polyhomoarginine comprising 6-26 homoarginine residues may be able to permeate physiological barriers more effectively, thereby facilitating efficient and fast treatment of an infection.

Further advantageously, a lower concentration of polyhomoarginine comprising 6-26 homoarginine residues may be required to treat an infection compared to a conventional antimicrobial agent.

More advantageously, the polyhomoarginine comprising 6-26 homoarginine residues may elicit rapid activity to effectively inhibit microbials such as fungi and bacteria, while being safe for use in mammals.

More advantageously, the activity of the polyhomoarginine comprising 6-26 homoarginine residues may not be abrogated by proteases, making them effective for use in physiological environments. More advantageously, the polyhomoarginine comprising 6-26 homoarginine residues may act to treat infections by a different mechanism to that of known antimicrobial agents such as natamycin.

Advantageously, the polyhomoarginine comprising 6-26 homoarginine residues may be safe for use in treatment of infections of the eye, where the tissue may be highly innervated and sensitive.

More advantageously, administration of the polyhomoarginine comprising 6-26 homoarginine residues may not cause any adverse effects to an animal infection model.

In another aspect, there is provided a method of treating an infection, wherein the infection is a fungal infection, a bacterial infection or a combination thereof, comprising administering a polyhomoarginine comprising 6-26 homoarginine residues or a pharmaceutically acceptable salt thereof to a subject.

In another aspect, there is provided a polyhomoarginine comprising 6-26 homoarginine residues or a pharmaceutically acceptable salt thereof, for use in treating an infection, wherein the infection is a fungal infection, a bacterial infection or a combination thereof.

In another aspect, there is provided an in vitro method of treating an infection, wherein the infection is a fungal infection, a bacterial infection or a combination thereof, comprising the step of administering a polyhomoarginine comprising 6-26 homoarginine residues or a pharmaceutically acceptable salt thereof, to a cell infected with bacteria and/or fungi.

In another aspect, there is provided the use of polyhomoarginine comprising 6-26 homoarginine residues or a pharmaceutically acceptable salt thereof, in a formulation, wherein the polyhomoarginine is present at a concentration in the range of 50 μg/mL to 50 mg/mL and the formulation comprises a pharmaceutically acceptable carrier.

Advantageously, the 6-26 homoarginine residues or a pharmaceutically acceptable salt thereof may be readily soluble in a solvent such as an aqueous buffer.

More advantageously, the 6-26 homoarginine residues or a pharmaceutically acceptable salt thereof may be stable in autoclaving conditions, making them suitable for use in sterilized formulations.

In another aspect, there is provided a contact lens comprising polyhomoarginine comprising 6-26 homoarginine residues or a pharmaceutically acceptable salt thereof.

In another aspect, there is provided the use of the contact lens as defined above, in the manufacture of a medicament for the treatment of an infection, wherein the infection is a fungal infection, a bacterial infection or a combination thereof.

Advantageously, the use of contact lenses may enhance the bioavailability of the polyhomoarginine compared to an eye drop application which may in turn minimise the frequency of application of the polyhomoarginine, and increase patient compliance and decrease drug wastage.

In another aspect, there is provided a method of treating an infection, wherein the infection is a fungal infection, a bacterial infection or a combination thereof, comprising applying the contact lens as defined above to an eye of a subject.

In another aspect, there is provided the contact lens as defined above, for use in treating an infection, wherein the infection is a fungal infection, a bacterial infection or a combination thereof.

### Definitions

The following words and terms used herein shall have the meaning indicated:

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Detailed Disclosure of Optional Embodiments

There is provided the use of polyhomoarginine (PHA) comprising 6-26 homoarginine residues or a pharmaceutically acceptable salt thereof, in the manufacture of a medicament for the treatment of an infection, wherein the infection is a fungal infection, a bacterial infection or a combination thereof.

The polyhomoarginine may comprise 6-26, 6-10, 6-14, 6-18, 6-22, 10-14, 10-18, 10-22, 10-26, 14-18, 14-22, 14-26, 18-22, 18-26 or 22-26 homoarginine residues.

The polyhomoarginine may comprise 8-15 homoarginine residues, preferably 10 homoarginine residues or may comprise a mixture of homoarginine residues of different residue numbers. The polyhomoarginine may comprise a mixture of 6, 10, and 15 homoarginine residues at equal weight ratios. For example, the polyhomoarginine may comprise 0.33% (w/w) each of 6, 10 and 15 homoarginine residues.

For example, polyhomoarginine comprising 10 residues may be referred to as PHA 10. PHA 10 may comprise an average of 10 homoarginine residues, and may comprise, in addition to polyhomoarginines having 10 residues, other polyhomoarginines having 8 to 12 residues. PHA 10 may be considered a low molecular weight polyhomoarginine comprising an average of 10 homoarginine residues.

The polyhomoarginine may have an average molecular weight in the range of about 2,000 to about 6,000, about 2,000 to about 3,000, about 2,000 to about 4,000, about 2,000 to about 5,000, about 3,000 to about 4,000, about 3,000 to about 5,000, about 3,000 to about 6,000, about 4,000 to about 5,000, about 4,000 to about 6,000 or about 5,000 to about 6,000 Dalton (Da).

The polyhomoarginine may comprise L-homoarginine and/or D-homoarginine. The polyhomoarginine may comprise L-homoarginine. The polyhomoarginine may comprise D-homoarginine. The polyhomoarginine may comprise L-homoarginine and D-homoarginine.

The polyhomoarginine may be optically pure.

The polyhomoarginine may consist only of L-homoarginine.

By pharmaceutically acceptable salt, it is meant those salts which, within the scope of sound medical judgement, are suitable for use in contact with the tissues of humans and lower animals without undue toxicity, irritation, allergic response and any combination thereof, and are commensurate with a reasonable benefit/risk ratio. Pharmaceutically acceptable salts are well known in the art.

For instance, suitable pharmaceutically acceptable salts of polyhomoarginine according to the present disclosure may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the polyhomoarginine of the invention. Suitable pharmaceutically acceptable salts of the polyhomoarginine of the present disclosure therefore include acid addition salts.

S. M. Berge et al. describe pharmaceutically acceptable salts in detail in J. Pharmaceutical Sciences, 1977, 66:1-19. The salts can be prepared in situ during the final isolation and purification of the polyhomoarginine of the invention, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and any combination thereof. Representative alkali or alkaline earth metal salts include sodium, lithium potassium, calcium, magnesium, and any combination thereof, as well as non-toxic ammonium, quaternary ammonium, and amine cations, including, but not limited to ammonium, tetramethylammonium, tetraethylammonium, methylamine, dimethylamine, trimethylamine, triethylamine, ethylamine, triethanolamine and any combination thereof.

The fungal infection may be caused by a fungus selected from the group consisting of Candida species, Fusarium species, Aspergillus species, Cryptococcus species and any mixture thereof.

The fungal infection may be caused by a fungus selected from the group consisting of C. albicans, C. parapsilosis, C. tropicalis, C. guillermondii, C. krusei, F. solani, F. dimerum, F. oxysporum, F. sacchari, and F. verticillioides, F. polyphialidicum, A. flavus, A. effusus, A. tamarii, A. sydowii, A. protuberus and A. terreus and any mixture thereof.

The bacterial infection may be caused by gram-positive bacteria and/or gram-negative bacteria. The bacterial infection may be caused by gram-positive bacteria. The bacterial infection may be caused by gram-negative bacteria. The bacterial infection may be caused by gram-positive bacteria and gram-negative bacteria.

The bacterial infection may be caused by a bacteria selected from the group consisting of Staphylococcus species, Streptococcus species, Pseudomonas species, Enterobacteriaceae, and any mixture thereof.

The bacterial infection may be caused by a bacteria selected from the group consisting of S. aureus, Methicillin-resistant S. aureus, S. epidermidis, S. pneumoniae, P. aeruginosa, and any mixture thereof.

The infection may further comprise an infection caused by an amoeba, preferably Acanthamoeba species.

The infection may comprise an infection caused by:
fungus
bacteria
a combination of fungus and bacteria
a combination of fungus and amoeba
a combination of bacteria and amoeba; or
a combination of fungus, bacteria and amoeba.

The infection may be an infection of the skin.

The infection may be an infection of the eye.

The infection may be an infection of the cornea of the eye.

The infection may be keratitis.

Convenient modes of administration include injection (subcutaneous, intravenous, etc.), transdermal application, topical creams or gels or powders. Depending on the route of administration, the formulation may be treated with a material to protect the formulation from the action of enzymes, acids and other natural conditions which may inactivate the therapeutic activity of the polyhomoarginine.

Dispersions of the polyhomoarginine according to the invention may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical formulations suitable for injection include sterile aqueous solutions (where water soluble) or dispersions and sterile powders for the extemporaneous preparation of sterile injectable solutions or dispersions. Ideally, the formulation is stable under the conditions of manufacture and storage and may include a preservative to stabilise the formulation against the contaminating action of microorganisms such as bacteria and fungi.

The polyhomoarginine may be administered topically, intrastromally, intracamerally, or by subconjunctival injection.

In one embodiment, the polyhomoarginine may be administered by injection. In the case of injectable solutions, the carrier can be a solvent or dispersion medium containing, for example, water, ethanol, polyol (for example, glycerol, propylene glycol, and liquid polyetheylene glycol, and any combination thereof), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and any combination thereof. In many cases, it may be preferable to include isotonic agents, for example, sugars, polyalcohols such as mannitol, sorbitol, sodium chloride in the formulation. Prolonged absorption of the injectable formulation can be brought about by including in the formulation an agent which delays absorption, for example, aluminium monostearate and gelatin.

Sterile injectable solutions can be prepared by incorporating the analogue in the required amount in an appropriate solvent with one or a combination of ingredients enumerated above, as required. followed by filtered sterilisation. Generally, dispersions are prepared by incorporating the analogue into a sterile vehicle which contains a basic dispersion medium or excipient and the required other ingredients from those enumerated above. The excipient may be a polypropylene glycol, polyvinyl alcohol, a carbomer, polycarbophil, a polyoxyethlene-polyoxypropylene block copolymer, hydroxy methylcellulse, carboxy methyl cellulose, hydroxy propyl methyl cellulose, hdroxy ethyl cellulse, dextran, gellan gum, guar gum or any two or more of the above excipients.

The polyhomoarginine may be administered topically in the form of a solution, suspension, emulsion, ointment, a cream, a gel or a sustained release vehicle, such as an ocular insert.

Further, it will be apparent to one of ordinary skill in the art that the optimal course of treatment, such as the number of doses of the polyhomoarginine or formulation of the invention given per day for a defined number of days, can be ascertained using convention course of treatment determination tests.

The polyhomoarginine may be administered in a formulation having a concentration in the range of about 50 μg/mL to about 50 mg/mL, about 50 μg/mL to about 100 μg/mL, about 50 μg/mL to about 200 μg/mL, about 50 μg/mL to about 500 μg/mL, about 50 μg/mL to about 1 mg/mL. about 50 μg/mL to about 2 mg/mL, about 50 μg/mL to about 5 mg/mL, about 50 μg/mL to about 10 mg/mL, about 50 μg/mL to about 20 mg/mL, about 100 μg/mL to about 200 μg/mL, about 100 μg/mL to about 500 μg/mL, about 100 μg/mL to about 1 mg/mL, about 100 μg/mL to about 2 mg/mL, about 100 μg/mL to about 5 mg/mL, about 100 μg/mL to about 10 mg/mL, about 100 μg/mL to about 20 mg/mL, about 100 μg/mL to about 50 mg/mL, about 200 μg/mL to about 500 μg/mL, about 200 μg/mL to about 1 mg/mL, about 200 μg/mL to about 2 mg/mL, about 200 μg/mL to about 5 mg/mL, about 200 μg/mL to about 10 mg/mL, about 200 μg/mL to about 20 mg/mL, about 200 μg/mL to about 50 mg/mL, about 500 μg/mL to about 1 mg/mL, about 500 μg/mL to about 2 mg/mL, about 500 μg/mL to about 5 mg/mL, about 500 μg/mL to about 10 mg/mL, about 500 μg/mL to about 20 mg/mL, about 500 μg/mL to about 50 mg/mL, about 1 mg/mL to about 2 mg/mL, about 1 mg/mL to about 5 mg/mL, about 1 mg/mL to about 10 mg/mL. about 1 mg/mL to about 20 mg/mL, about 1 mg/mL to about 50 mg/mL, about 2 mg/mL to about 5 mg/mL, about 2 mg/mL to about 10 mg/mL, about 2 mg/mL to about 20 mg/mL, about 2 mg/mL to about 50 mg/mL, about 5 mg/mL to about 10 mg/mL, about 5 mg/mL to about 20 mg/mL. about 5 mg/mL to about 50 mg/mL, about 10 mg/mL to about 20 mg/mL, about 10 mg/mL to about 50 mg/mL or about 20 mg/mL to about 50 mg/mL.

The polyhomoarginine may be administered 1 to 12 times, 1 to 2 times, 1 to 5 times, 1 to 10 times, 2 to 5 times, 2 to 10 times, 2 to 12 times, 5 to 10 times, 5 to 12 times or 10 to 12 times per day.

The use may comprise a further antimicrobial agent.

The further antimicrobial agent may be an antibacterial agent or an antifungal agent.

The further antimicrobial agent may be selected from the group consisting of amphotericin B. natamycin, fluconazole, ketaconazole, voriconazole, miconazole, luliconazole and any mixture thereof.

There may be synergistic effects between the polyhomoarginine comprising 6-26 homoarginine residues or a pharmaceutically acceptable salt thereof, and the further antimicrobial agent.

There is also provided a method of treating an infection, wherein the infection is a fungal infection, a bacterial infection or a combination thereof, comprising administering a polyhomoarginine comprising 6-26 homoarginine residues or a pharmaceutically acceptable salt thereof to a subject.

There is also provided a polyhomoarginine comprising 6-26 homoarginine residues or a pharmaceutically acceptable salt thereof, for use in treating an infection, wherein the infection is a fungal infection, a bacterial infection or a combination thereof.

There is also provided an in vitro method of treating an infection, wherein the infection is a fungal infection, a bacterial infection or a combination thereof, comprising the step of administering a polyhomoarginine comprising 6-26 homoarginine residues or a pharmaceutically acceptable salt thereof, to a cell infected with bacteria and/or fungi.

In accordance with the present disclosure, when used for the treatment or prevention of an infection, polyhomoarginine of the present disclosure may be administered alone. Alternatively, the polyhomoarginine may be administered as a pharmaceutical, veterinarial, agricultural, or industrial formulation which comprises at least one polyhomoarginine according to the disclosure.

There is also provided the use of polyhomoarginine comprising 6-26 homoarginine residues or a pharmaceutically acceptable salt thereof, in a formulation, wherein the polyhomoarginine is present at a concentration in the range of 50 μg/mL to 50 mg/mL and the formulation comprises a pharmaceutically acceptable carrier.

The polyhomoarginine may be present in the at a concentration in the range of about 50 μg/mL to about 50 mg/mL, about 50 μg/mL to about 100 μg/mL, about 50 μg/mL to about 200 μg/mL. about 50 μg/mL to about 500 μg/mL, about 50 μg/mL to about 1 mg/mL, about 50 μg/mL to about 2 mg/mL, about 50 μg/mL to about 5 mg/mL, about 50 μg/mL to about 10 mg/mL, about 50 μg/mL to about 20 mg/mL, about 100 μg/mL to about 200 μg/mL, about 100 μg/mL to about 500 μg/mL, about 100 μg/mL to about 1 mg/mL, about 100 μg/mL to about 2 mg/mL, about 100 μg/mL to about 5 mg/mL, about 100 μg/mL to about 10 mg/mL, about 100 μg/mL to about 20 mg/mL, about 100 μg/mL to about 50 mg/mL, about 200 μg/mL to about 500 μg/mL, about 200 μg/mL to about 1 mg/mL, about 200 μg/mL to about 2 mg/mL, about 200 μg/mL to about 5 mg/mL, about 200 μg/mL to about 10 mg/mL, about 200 μg/mL to about 20 mg/mL, about 200 μg/mL to about 50 mg/mL, about 500 μg/mL to about 1 mg/mL, about 500 μg/mL to about 2 mg/mL, about 500 μg/mL to about 5 mg/mL, about 500 μg/mL to about 10 mg/mL, about 500 μg/mL to about 20 mg/mL, about 500 μg/mL to about 50 mg/mL, about 1 mg/mL to about 2 mg/mL, about 1 mg/mL to about 5 mg/mL, about 1 mg/mL to about 10 mg/mL, about 1 mg/mL to about 20 mg/mL, about 1 mg/mL to about 50 mg/mL, about 2 mg/mL to about 5 mg/mL, about 2 mg/mL to about 10 mg/mL, about 2 mg/mL to about 20 mg/mL, about 2 mg/mL to about 50 mg/mL, about 5 mg/mL to about 10 mg/mL, about 5 mg/mL to about 20 mg/mL, about 5 mg/mL to about 50 mg/mL, about 10 mg/mL to about 20 mg/mL, about 10 mg/mL to about 50 mg/mL or about 20 mg/mL to about 50 mg/mL.

The formulation may comprise a further antimicrobial agent. The further antimicrobial agent may be the same as the further antimicrobial agent defined above.

The language “pharmaceutically acceptable carrier” is intended to include solvents, dispersion media, coatings, anti-bacterial and anti-fungal agents, isotonic and absorption delaying agents, and any combination thereof. The use of such media and agents for pharmaceutically active substances is well known in the art. Except insofar as any conventional media or agent is incompatible with the polyhomoarginine, use thereof in the formulation and methods of treatment and prophylaxis is contemplated.

Preferably, the formulation may further include a suitable buffer to minimise acid hydrolysis. Suitable buffer agent agents are well known to those skilled in the art and include, but are not limited to, phosphates, citrates, carbonates and mixtures thereof.

The formulation may be in the form of a solution, suspension, emulsion, ointment, a cream or a gel. The formulation may be an eye drop formulation.

The formulation may be an eye drop formulation.

The formulation may comprise an aqueous buffer. The formulation may have a pH in the range of about 4.5 to about 8, about 4.5 to about 5, about 4.5 to about 6, about 4.5 to about 7, about 5 to about 6, about 5 to about 7, about 5 to about 8, about 6 to about 7, about 6 to about 8 or about 7 to about 8. The formulation may comprise phosphate buffered saline (PBS) at physiological pH such as pH7.4.

Also included in the scope of this invention are delayed release formulations.

There is also provided a contact lens comprising polyhomoarginine comprising 6-26 homoarginine residues or a pharmaceutically acceptable salt thereof.

The contact lens may be any ionic high water content contact lens. The contact lens may be a soft contact lens. The contact lens may comprise 2-hydroxyethyl methacrylate, methacrylic acid, polyvinylpyrrolidone and any mixture thereof. The contact lens may be a hydrogel. The contact lens may be selected from the group consisting of etafilcon A, ocufilcon D, vifilcon A and any mixture thereof.

The water content of the contact lens may at least partially be replaced with polyhomoarginine comprising 6-26 homoarginine residues or a pharmaceutically acceptable salt thereof.

The water content of the contact lens may at least partially be replaced with a formulation as defined above.

The formulation may comprise polyhomoarginine comprising 6-26 homoarginine residues at a concentration in the range of about 0.001% w/v to about 0.1% w/v, about 0.001% w/v to about 0.002% w/v, about 0.001% w/v to about 0.005% w/v, about 0.001% w/v to about 0.01% w/v, about 0.001% w/v to about 0.02% w/v, about 0.001% w/v to about 0.05% w/v, about 0.002% w/v to about 0.005% w/v, about 0.002% w/v to about 0.01% w/v, about 0.002% w/v to about 0.02% w/v, about 0.002% w/v to about 0.05% w/v, about 0.002% w/v to about 0.1% w/v, about 0.005% w/v to about 0.01% w/v, about 0.005% w/v to about 0.02% w/v, about 0.005% w/v to about 0.05% w/v, about 0.005% w/v to about 0.1% w/v, about 0.01% w/v to about 0.02% w/v, about 0.01% w/v to about 0.05% w/v, about 0.01% w/v to about 0.1% w/v, about 0.02% w/v to about 0.05% w/v, about 0.02% w/v to about 0.1% w/v or about 0.05% w/v to about 0.1% w/v.

The formulation may be in PBS (pH 7.0).

There is also provided the use of the contact lens as defined above, in the manufacture of a medicament for the treatment of an infection, wherein the infection is a fungal infection, a bacterial infection or a combination thereof.

There is also provided a method of treating an infection, wherein the infection is a fungal infection, a bacterial infection or a combination thereof, comprising applying the contact lens as defined above to an eye of a subject.

There is also provided the contact lens as defined above, for use in treating an infection, wherein the infection is a fungal infection, a bacterial infection or a combination thereof.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 is a set of graphs showing the cell viability of PHA polymers of various chain length (PHA10, PHA30, PHA50, PHA 100, PHA2500) for a) HaCaT cells and b) primary human dermal fibroblast cells. Each point in the figure represents mean±s.d., from quadruplicates.

FIG. 2 is a set of graphs showing time-kill kinetics of PHA 10 against A) F. solani ATCC 46492 (MIC=2 mg/mL) and B) F. solani ATCC 62877 strains (MIC=4 mg/mL).

FIG. 3 is a set of graphs showing A) DiSC₃-5 efflux assay showing rapid depolarization of fungal cell membrane upon addition of PHA 10; and B) Determination of extracellular ATP upon addition of PHA 10 to F. solani cells after a 6 hour exposure.

FIG. 4 is a set of graphs showing antifungal activity of PHA of various chain length in the presence of A) trypsin and B) MMP-9 against F. solani 46492 (MIC=2 mg/mL) and B) F. solani 62877 (MIC=4 mg/mL) strains.

FIG. 5 is a set of graphs showing antifungal activity of PHA 10 in the presence of tear fluid against A) F. solani 46492 (MIC=2 mg/mL) and B) F. solani 62877 (MIC=4 mg/mL) strains.

FIG. 6 is a set of graphs and micrographs showing the cell viability of F. solani hyphal after treatment with varying concentration of PHA polymers of various chain length. A) is a graph summarising the cell viability of F. solani hyphal, and B) to I) are optical micrographs (63×) showing the F. solani hyphal after treatment with B) PHA 10, C) PHA 30, D) PHA 50, E) PHA 100, F) PHA 250, G) PHA400, H) Natamycin and I) control.

FIG. 7 is a set of images showing A) Fluorescent slit lamp biomicroscopy images of the progression of wound healing after topical instillation of vehicle, PHA 10 or natamycin on days 0, 1, 2 and 3; and B) Graph showing the change in wound area after topical instillation for various groups. A total of 6 eyes were used per group.

FIG. 8 is a set of images and graphs showing A) Slit lamp biomicroscopy images of the efficacy of vehicle, PHA 10 and natamycin in a Fusarium keratitis model on day 1 and day 7. F. solani ATCC 46492 was used in the study. B) Graph showing change in wound area in a Fusarium infected rabbit after treatment with various groups. *, p≤0.05; *** and p≤0.001 by Mann-Whitney test, and C) Graph showing fungal bioburden in the excised cornea after treatment (4 times/day for 7 days) with various groups. **, p≤0.01 by Mann-Whitney test.

FIG. 9 is a set of images and graphs showing A) A schematic diagram showing the experimental strategy to assess the antifungal efficacy of PHA 10 and comparator antifungal, voriconazole in a mice model of Candida keratitis. B) Slit lamp biomicroscopy images (top panel) and AS-OCT images (bottom panel) showing the efficacy of vehicle, PHA 10 and voriconazole at 6 hours post inoculation (p.i.), as well as after 14 doses of treatment with vehicle or antifungals.in a Candida keratitis model. C. albicans SC 5314 was used in the study. C) and D) Histological examination of eyes treated with C) Voriconazole and D) PHA10. Scale bar represents 100 μm.

FIG. 10 is a set of images showing slit lamp examination (A) and AS-OCT (B) images in a mice model of Candida keratitis. Mouse eyes were infected with Candida albicans for 6 hours (A1, B1, A3 and B3) and treated with 14 doses of PHA 10 (A2 and B2) or PHA 30 (A4 and B4).

FIG. 11 is a graph showing the fungal burden in a mouse model of Candida keratitis after treatment with PHA10, PHA30 or vehicle only.

FIG. 12 is a set of images showing A) AS-OCT images that describe the analysis of various parameters that show the extent of anterior chamber inflammation before and after treatment in a Candida keratitis model. B) A graph showing the presence of anterior chamber cells expressed in terms of Grade 0-Grade 4; C) A graph showing anterior chamber depth; D) A graph showing central corneal thickness; E) a graph showing iris-cornea angle and f) A graph showing fungal burden in the eye after treatment with various groups.

FIG. 13 is a set of images showing slit lamp examination (A, top panel) and AS-OCT (B, bottom panel) images of P. aeruginosa infected eyes that are (1) untreated or treated with (2) PHA10, (3) Tobradex or (4) Tobrex. Tobradex (3) contains 0.3% tobramycin and 0.1% corticosteroid, dexamethasone. Tobrex (4) contains 0.3% tobramycin. P. aeurinosa infected eyes were treated with topical administration (4 times/day) for 3 days for the various treatment groups.

FIG. 14 is a graph showing bacterial viability after treatment with PHA10. Three out of the five eyes (i.e. 60%) that received PHA10 responded to the treatment and displayed a reduction in bacterial burden when compared to untreated control.

FIG. 15 is a set of images showing A) a schematic diagram showing how PHA 10 may be incorporated in a Etafilcon contact lens and B) release profile of PHA 10 from Etafilcon contact lens.

### EXAMPLES

Non-limiting examples of the invention will be further described in greater detail by reference to specific Examples, which should not be construed as in any way limiting the scope of the invention.

### Materials and Methods

### Materials

All the Fusarium solani strains used in this study were purchased from American Type Culture Collection (Manassas, Virginia, USA). Roswell Park Memorial Institute (RPMI-1640) medium was purchased from M/s. Biocon Solutions Pte Ltd., Singapore. Polyhomoarginine (PHA) of various chain lengths (10-400 residues) were purchased from M/s Alamanda Polymers, Inc (Huntsville, Alabama, USA) and used as-purchased for the experiments. MTS (3-(4, 5-dimethylthiazol-2-yl)-5-(3-carboxymethoxyphenyl)-2-(4-sulfophenyl)-2H-tetrazolium) was purchased from Promega Corporation (Madison, Wisconsin, USA). All the mammalian cell culture reagents were obtained from Life Technologies Corporation, Singapore. New Zealand White rabbits (2-3.0 kg) were purchased from Bio Conquest Pte Ltd, Singapore and Covance Research Products Inc, Denver, Pennsylvania, USA. C57BL/6 mouse were purchased from InVivos Pte Ltd., Singapore. 1% voriconazole was obtained as 1% formulation from the pharmacy at the Singapore National Eye Centre. Xylazine was purchased from MP biomedicals Asia Pacific Pte Ltd., Singapore. Fluorescamine, MMP-9, trypsin, diS-C3-5, and lignocaine hydrochloride was purchased from Merk (Singapore) Pte Ltd.

### Antifungal Susceptibility Experiments

Antifungal susceptibility test was performed based on the CLSI M38 A2 guidelines to determine the minimum inhibitory concentration (MIC). Two-fold serial dilution of the peptides and polymers in Roswell Park Memorial Institute (RPMI-1640) medium was performed on a 96-well plate to obtain varying concentration of peptides and polymers. Spore suspensions were adjusted to an optical density (OD₅₃₀) of 0.15 to 0.17 at 530 nm using an Infinite M200 microplate reader (Tecan Group Ltd., Männedorf, Switzerland), further diluted 1:50 in RPMI-1640 and inoculated onto the peptide and polymer treated 96-well plate. The final inoculum size was between 0.4×10⁴ to 5×10⁴ spores/ml. Wells inoculated without peptide or polymer addition and wells containing only broth served as positive and negative control, respectively. Plates were then incubated for 48 hours at 30° C. and MIC₉₀ was determined at OD₅₃₀ using the microplate reader and by visual observation. MIC₉₀ was defined as the concentration that caused ≥90% of the fungal growth inhibition.

### Fungal Hyphal Inhibition Assay

Spore suspension was adjusted to 5×10⁴ spores/ml in RPMI-1640 medium and inoculated onto a 96-well plate. Plates were incubated for 16 hours at 30° C. for hyphal formation and then exposed to various concentration of polymers in 2-fold dilution (0.5-128 mg/mL). Plates were then incubated for 2 hours at room temperature and 20 μl of MTS (3-(4, 5-dimethylthiazol-2-yl)-5-(3-carboxymethoxyphenyl)-2-(4-sulfophenyl)-2H-tetrazolium) (Promega Corporation, Madison, Wisconsin, USA) was added into each well. After incubation at room temperature for another 1-4 hours, absorbance was measured using the microplate reader at 490 nm and the % viable cells was determined. Concentration of polymer required for 50% hyphal inhibition was then determined (IC₅₀), by Graphpad Prism 8.0. Morphological effects exerted by the peptide and polymers on the fungal hyphal were observed using an Axio Observer Z1(ZEISS, Oberkochen, Germany) after exposing the hyphal to the peptides and polymers for 24 hours.

### Cytotoxicity of PHAs for HaCaT and Primary Human Dermal Fibroblasts

Primary human dermal fibroblasts/corneal fibroblasts and human corneal epithelial/HaCaT cell lines were cultured in Gibco™ DMEM or DMEM/F12 (1:1) containing 10% FBS and 1% Penstrep and incubated in Nunc-75 cm² tissue culture flasks (Thermo Fisher Scientific, Waltham, Massachusetts, USA) in a humidified chamber at 37° C. supplied with 5% CO₂. For cytotoxicity assays, overnight culture in 96 well plate in triplicates at a seeding density of 3×10³ cells per well were treated with various concentration of PHAs (0.005 μg/mL to 1 mg/mL). After 24 hours of incubation at 37° C., cell viability was determined using CellTiter 96® Aqueous One solution cell proliferation assay kit (Promega, WI, USA) according to the manufacturer's instructions. The mean±s.d., value was plotted and fit into sigmoidal function using Graphpad Prism 8.0 to determine the concentration of polymer that caused 50% cytotoxicity (CC₅₀). From IC₅₀ and CC₅₀, selectivity index (SI) is calculated as CC₅₀/IC₅₀ and tabulated.

### Determination of Zeta Potential

Polymers were diluted in Phosphate Buffered Saline (PBS) pH 7.2 to a final concentration of 50 μM. Zeta Potential was measured using a Litesizer 500 (Anton Paar, Graz, Austria). Mean±s.d., from 4 independent experiments was tabulated.

### Time-Kill Kinetics Assay

1 ml stock solution of the polymer were prepared in RPMI buffer adjusted to a final concentration of 4×, 2×, and 1× minimum inhibitory concentration (MIC) values. 1 mL of fungal inoculum (2−4×10⁴ CFU/mL) was added to each solution and incubated at 30° C. for 48 hours. 100 μL was withdrawn, serially diluted with PBS to obtain various dilution factors (10¹, 10², 10³), and cultured on separate Sabouraud dextrose agar plates in duplicates. All the plates were incubated at 30° C. for 72 hours. The surviving colonies were enumerated and reported as colony-forming units per ml (CFU/ml) after incubation.

### Disc3-5 Assay

Changes in membrane potential of F. solani upon addition of PHA10 were monitored by the release of potential sensitive probe diS-C3-5 dye from intact fungal cells. Briefly. 1 ml of overnight grown fungal cells in 5 mM HEPES buffer (pH 7.0) was mixed with 10 μM diS-C3-5 and incubated at 37° C. for 1-2 h in a thermoshaker. 800 μL of the dye-loaded cell suspension was transferred to a stirred quartz cuvette and the change in fluorescence intensity was monitored at an emission wavelength (λₑₘ) of 670 nm (excitation wavelength. 622 nm) using a Quanta Master spectrofluorimeter (Photon Technology International, Birmingham, New Jersey, USA). The excitation and emission bandwidths were set at 1 and 2 nm, respectively. Once a constant fluorescence level was achieved. 10 μL of concentrated peptide solution in HEPES buffer was added so that the final concentration of polymer was 1-8×MIC.

### Adenosine Triphosphate (ATP) Efflux Assay

The extracellular ATP levels upon challenging F. solani with PHA 10 was determined by incubating the microbial cells (OD₅₃₀≈0.2) with various concentrations of the polymer (1×-8× MIC) for 6 hours at 37° C. with orbital shaking. Each tube was then centrifuged at 5.000 g for 5 minutes. Then. 225 μL of boiling TE buffer (50 mM Tris, 2 mM EDTA, pH 7.8) was added to the 25 μl of the supernatant and mixed well. This mixture was boiled again for another 2 minutes and stored at 4° C. until further examination. 100 μL of a luciferin-luciferase ATP assay mixture was added to 100 μL of the supernatant and luminescence was monitored using the Infinite M200 microplate reader (Tecan Group Ltd., Männedorf, Switzerland). The extracellular ATP concentration was determined from calibration curve obtained by ATP assay kit (Molecular Probes, Eugene, Oregon, USA) as per the manufacturer's instruction.

### Antifungal Activities of PHAs in the Presence of MMP-9, Trypsin and in Tear Fluid

Antifungal activity of PHA against two F. solani strains was determined after exposure to several proteases. Each polymer was incubated with trypsin and MMP-9 in separate experiments (protease:peptide ratio=1:100) and incubated for 1 hour at 37° C. 1 mL of fungal inoculum (×10⁴ CFU/mL) was then added into each solution and incubated for 48 hours at 30° C. The final concentration of PHA was maintained at 4×MIC. Culture alone served as the positive control. After incubation, 100 μl was withdrawn from each solution and serially diluted with PBS to obtain various dilution factors (10-1, 10-2, 10-3). 100 μl aliquots of the dilutions were cultured on potato dextrose agar plates in duplicates. Plates were incubated for 72 hours at 30° C. and surviving colonies were enumerated. Three independent replicates were performed for the experiment. This procedure was repeated with MMP-9 instead of trypsin. For MMP-9, culture alone and culture with MMP-9 were used as positive control. After which, polymers were exposed to F. solani for 48 hours at 30° C. and fungal viability was determined.

To assess the antifungal activity in tea fluid (TF). PHA was dissolved in RPMI buffer. 10 μl of each PHA solution was incubated with 10 μl of rabbit TF for 1 hour at 37° C. 180 μl of fungal inoculum (×10⁴ CFU/mL) was then added into each solution and incubated for 48 hours at 30° C. The final concentrations of PHA remained 1×-32×MIC. For positive controls, culture alone (without TF) and culture with 50% TF were used. Tenfold serial dilutions and plating were performed as above to determine fungal viability.

### Rabbit Model of Corneal Wound Healing

New Zealand white rabbits weighing 2.5 kg were used for studying corneal wound healing. A 6 mm diameter wound was created on corneal surface using a trephine to mark the wound area and the corneal epithelium was debrided with the dull-bladed knife without damaging deeper corneal layers. The respective drugs and control vehicle were instilled on the ocular surface four times daily during day cycle. The wound defect area was monitored after fluorescein staining and imaged by a new-generation NS-2D Zoom clinical slit lamp (Righton, Japan) with the aid of Minims fluorescein sodium eye drops (Bausch and Lomb, Bridgewater, New Jersey, USA, 2% wt/vol). The area of corneal abrasion was determined by using Image J analysis. Corneal thickness was also measured perpendicular to the anterior corneal surfaces by AS-OCT (RTvue, Optovue, Fremont, California, USA).

### Rabbit Model of Fusarium Keratitis

Rabbits were immunosuppressed systemically by cortisone acetate 100 mg/kg and cyclophosphamide 100 mg/kg subcutaneously 2 days before infection and 3 days after infection. Local immunosuppression was achieved by subconjunctival injection of dexamethasone 0.8 mg once/day for 5 days before infection. Ceftazidime (50 mg/kg) was given subcutaneously every day from the beginning of the systemic immunosuppression protocol. F. solani ATCC 46492 was used to infect by injecting an initial inoculum≈7×10⁵ colony-forming units (CFU). 15 μL of fungal spores midstromally by an insulin syringe with 31-G needle (BD, Franklin Lakes, New Jerse, USA). The right eye was infected while the left eye was conserved as untouched. All the treatments started at Days-Post-infection (DPI)-5. Fluorescent Slit-lamp photographs were taken at 0 DPT and 7 DPT to assess the treatment response by employing a new-generation NS-2D Zoom clinical slit lamp (Righton, Japan) with the aid of Minims fluorescein sodium eye drops (Bausch and Lomb, Bridgewater, New Jersey, USA, 2% wt/vol). Corneal edema and thickness were measured at 0 DPT. 2 DPT, 4 DPT, 6 DPT and 7 DPT by using AS-OCT (RTvue, Optovue, Fremont, California, USA). At the end of experiment, corneas were harvested for counting viable fungal counts in the excised cornea. Viable fungal counts difference between untreated control and PHA 10/natamycin were analyzed by the Kruskal-Wallis test (PASW statistic 18), and P value was set to be significant at 0.05.

### Mice Model of Candida Keratitis

C. albicans SC5314 were cultured on Sabouraud dextrose agar plates at 37° C. for 24-48 hours. Colonies were harvested and suspended in sterile saline to achieve a final concentration of about 1×10⁹ CFU/L. Pathogen-free wild type C57BL/6 mice weighing 20-24 g were used for the study. The animals were treated in compliance with the SingHealth IACUC guidelines (2022/SHS/1761) and the ARVO guidelines for animal experimentation. Prior to infection, mice underwent ocular examination using slit-lamp photography (SLP) and anterior segment optical coherence tomography (AS-OCT) to confirm the absence of corneal defects. To introduce cornea infection, mice were anesthetized with xylazine (10 mg/kg, Troy Laboratories, Glendenning, NSW, Australia) and ketamine (80 mg/kg. Parnell Laboratories, Sydney, NSW, Australia). The corneal epithelium was scratched with a sterile Beaver 6400 Mini-Blade under a dissecting microscope (Zeiss, Oberkochen, Germany, Stemi-2000C) to create a superficial wound. Topical anaesthesia (1-5% lignocaine hydrochloride) was applied, and the cornea was rinsed with sterile saline. Subsequently, 15 μL of the Candida suspension was topically applied to the abraded corneal surface.

Treatment commenced 6 hours post-infection with 15 μL doses administered topically at 30-minute intervals. Vehicle, the test compound (PHA 10, 0.3% w/v) or 1% voriconazole were applied eight times (from 2:00 p.m. to 6:00 p.m.) on day one, and six instillations were made (from 8:30 a.m. to 11:00 a.m.) on day two (a total number of 14 doses). All mice were monitored for ocular changes using SLP and AS-OCT. The images of the anterior chamber (AC) of the eyes were acquired using an RTVue RT-100 OCT system (Optovue, Fremont, California, USA). Mouse was positioned in front of the optical coherence tomography (OCT) machine with the eye aligned to the objective of the machine. A drop of 0.9% physiological saline was instilled to maintain cornea hydration and the OCT images were acquired. The captured images were then processed using RTVue XR (version 2018.1.1.85) (Optovue, Fremont, California, USA) to quantify different parameters. A perpendicular line was drawn across the central cornea to determine central corneal thickness (CCT). The AC depth (ACD) was calculated by measuring the distance between the posterior surface of the cornea to the anterior surface of the lens. By measuring the angle between the iris and cornea at the angle recess, angles of the eye were calculated. Using the OCT images, percentage of cells that appear as hyper reflective spots in the AC was graded and compared.

At 3 hours post last dose, mice were sacrificed to quantify the fungal burden in the infected eyes. The eyes were dissected and homogenized in sterile PBS using a Pellet pestles cordless motor (Z359971. Sigma-Aldrich, St. Louis, Minnesota, USA) with glass beads to ensure thorough homogenization. The resulting homogenates were then subjected to 10-fold serial dilutions in sterile PBS (10⁰ to 10⁵). 100 μL of each dilution was inoculated in duplicate onto Sabouraud dextrose agar (SDA) plates and incubated at 37° C. for 48 hours. Colonies were then counted, and the results were expressed as colony-forming units (CFU).

Whole mouse eye was enucleated and fixed in 10% neutral buffered formalin solution (Leica Surgipath, Leica Biosystems Richmond, Inc., Richmond, Illinois, USA) for 24 hours, dehydrated in increasing concentration of ethanol, clearance in xylene, and embedding in paraffin (Leica-Surgipath, Leica Biosystems Richmond, Inc., Richmond, Illinois, USA). Four-micron sections were cut with a rotary microtome (RM2255, Leica Biosystems Nussloch GmbH, Nussloch, Germany) and collected on POLYSINE™ microscope glass slides (Gerhard Menzel, Thermo Fisher Scientific, Newington, Connecticut, USA). The sections were dried in an oven of 37° C. for at least 24 hours. To prepare the sections for histopathological examination, the sections were heated on a 60° C. hot plate, deparaffinized in xylene and rehydrated in decreasing concentration of ethanol. A standard procedure for Hematoxylin and Eosin (H&E) was performed. A light microscope (NIS-Elements, Nikon ECLIPSE Ti. Tokyo, Japan) was used to examine the slides and the images were captured.

For testing PHA 30, the same experimental protocol as above was used, but with 0.3% (3 mg/mL) of PHA 30 and the starting inoculum size was 1×10⁶ CFU/uL.

### Mouse Model of Pseudomonas Keratitis

The in vivo antimicrobial efficacy of PHA 10 was examined using an established pre-clinical murine bacterial keratitis model. C57 BL/6 mice were used for the study. All mice (n=6 mice/group) were randomly allocated to each treatment group, namely phosphate-buffered saline (PBS; negative control), PHA 10, Tobrex (commercial preparations containing 0.3% tobramycin) or Tobradex (preparation containing 0.3% tobramycin and 0.1% corticosteroid, dexamethasone). Slit-lamp examination was performed to confirm the health of corneas before starting the animal trials. After the administration of general anaesthesia [with intraperitoneal injections of xylazine (10 mg/kg) and ketamine (80 mg/kg)], and topical anaesthesia (with proxymetacaine hydrochloride 0.5%), the central 2 mm corneal epithelium was gently removed with sterile mini-blades (Beaver. MA. USA), leaving the basal lamina intact. 10 μl of ˜5×10⁶ CFU/ml of PA9027 (ATCC) was applied topically onto the cornea and the lid was held shut for 1 minute. At 24 hours post-infection. 10 μl of ˜5×10⁶ CFU/ml of PA9027 (ATCC) was applied topically onto the cornea and the lid was held shut for 1 minute. At 24 hour post-infection. 10 μl of respective treatment was applied directly onto the infected corneas four times daily (3 hours apart) for 3 days (a total number of 12 doses). The eyes were monitored daily with slit-lamp biomicroscopic photography and anterior segment optical coherence tomography (AS-OCT). AS-OCT (RTVue, Optovue, Fremont, CA) was used to measure the corneal thickness perpendicular to the anterior corneal surface at baseline and post-infection.

### Contact Lens (CL) Loading and Release Kinetics of PHA10

2 ml soak solutions of 40 μg/ml PHA 10 in PBS (pH 7.0) which correspond to 10-20×MIC of the polymer were prepared. Controls containing only 2 ml PBS without polymer were also prepared. After removal from its original packaging, Etafilcon A −3.0D (low myopic) soft contact lenses (Johnson & Johnson Inc., Palm Beach Gardens, Florida, USA) were blotted dry on lint-free paper and transferred to vials containing 2 ml of polymer solution. The vials were then autoclaved at 110° C. for 18 minutes, then left to equilibrate at room temperature for 48 hours.

Spectrofluorometric Quantification of Polymer Released from CL

Fluorescamine in acetone (0.1% w/v) served as an amine marker to detect the PHA 10. A linear calibration method was used to quantitate the lens uptake and release kinetics study. 50 μL of sample was added to 50 μL of fluorescamine in a black 96-well plate. Fluorescence intensity (FI) was measured using EnSpire Multimode Plate Reader (PerkinElmer, Inc., Waltham, Massachusetts, USA) within one minute of adding fluorescamine to prevent signal loss. Excitation and emission wavelength were set at 390 nm and 475 nm respectively.

After autoclaving, CLs were removed and blotted dry on lint-free paper for release study while the remaining loading solution in the vials was used to determine polymer uptake by spectrofluorometric quantification. The concentration of this remaining solution was subtracted from the initial loading concentration to determine the polymer uptake concentration. Release kinetics of each CL immersed in 4 mL of PBS at room temperature was studied. At specified time points (0.5, 1, 2, 4, 6 and 24 hours), 50 μL of PBS was aliquoted for spectrofluorometric quantification and replaced with 50 μL of fresh PBS. Uptake and release were performed in triplicates and mean±s.d., values were reported.

### Example 1: Antifungal Efficacy

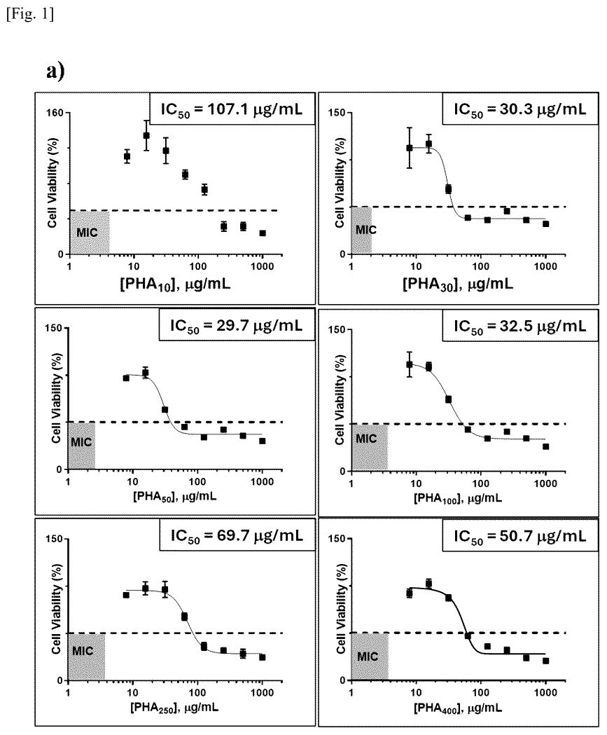

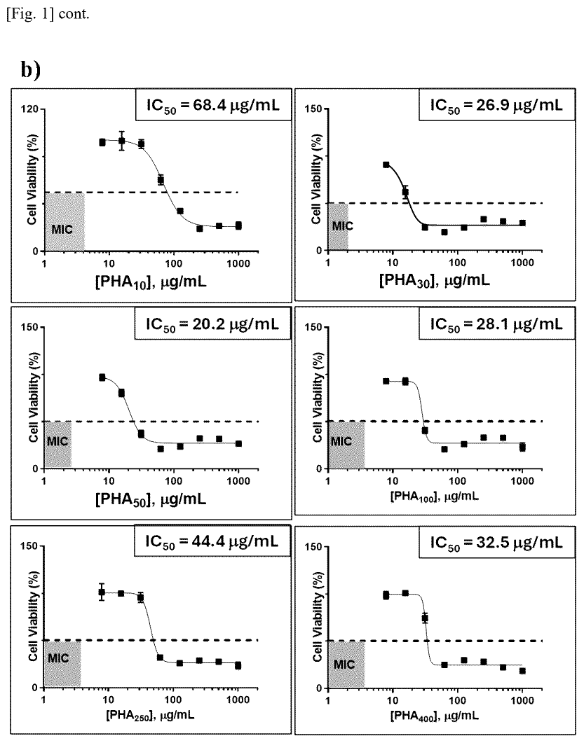

FIG. 1 shows the cell viability graphs of PHA polymers of various chain length in mammalian cells HacaT and HDF.

Table 1 below shows the antifungal inhibitory concentration and cytotoxicity for mammalian corneal cells of PHA of various chain length.

TABLE 1
Selectivity index of PHAs and Natamycin

| | IC₅₀ on F. | EC₅₀ | | SI | |
|---|---|---|---|---|---|
| | solani hyphal | FB^{a} | HCE^{b} | Fb^{ac} | HCE^{bd} |
| Compound | (mg/ml) | (mM) | (mM) | (mM) | (mM) |
| PHA 10 MW | 12.1 | 59.2 | 5.0 | 4.9 | 0.41* |
| 2,100 | | | | | |
| PHA 30 MW | 11.1 | <4^{e} | 1.5 | <0.4* | 0.14* |
| 6,200 | | | | | |
| PHA 50 MW | 11.4 | 5.4 | <4^{f} | 0.47* | <0.35* |
| 10,000 | | | | | |
| PHA 100 MW | 6.4 | <4^{e} | <4^{f} | <0.63* | <0.63* |
| 21,000 | | | | | |
| PHA 250 MW | 4.5 | <4^{e} | <4^{f} | <0.89* | <0.89* |
| 52,000 | | | | | |
| PHA 400 MW | 0.97 | <4^{e} | <4^{f} | <0.82* | <0.82* |
| 83,000 | | | | | |
| Natamycin | 8.4 | 51.8 | 31.2 | 6.17 | 3.71 |

^{a}Fb, human corneal fibroblast cells
^{b}HCE, human corneal epithelial cells
^{c}SI, Selectivity Index is calculated as EC50 on Fb/IC50 on F. solani hyphal
^{d}SI, Selectivity Index is calculated as EC50 on HCE/IC50 on F. solani hyphal
^{e}Lowest concentration of compound used for this study was unable to achieve 50% cell viability for Fb cells. IC50 is assumed to be lower than the lowest concentration that was used for this study.
^{f}Lowest concentration of compound used for this study was unable to achieve at least 50% cell viability for HCE cells. IC₅₀ is assumed to be lower than the lowest concentration that was used for this study.
*SI < 1

Table 2 shows the minimum inhibitory concentration (MIC) of PHA polymers of various chain length against F. solani strains.

TABLE 2
Susceptibility profile of Fusarium species to PHAs and natamycin
based on CLSI (Clinical and Laboratory Standards Institute) standard

| | Minimum inhibitory concentration (μg/ml) of | | | | | | |
|---|---|---|---|---|---|---|---|
| | PHA10 | PHA30 | PHA50 | PHA100 | PHA250 | PHA400 | |
| F. solani | (MW- | (MW- | (MW- | (MW- | (MW- | (MW- | |
| (ATCC strains) | 2,100) | 6,200) | 10,000) | 21,000) | 52,000) | 83,000) | Natamycin |
| 46492 | 2 | 2 | 2 | 4 | 4 | 4 | 8 |
| 3636 | 4 | 2 | 4 | 4 | 4 | 4 | 16 |
| 52628 | 4 | 2 | 2 | 4 | 4 | 4 | 16 |
| 62877 | 4 | 2 | 2 | 2 | 2 | 2 | 8 |
| 36031 | 4 | 8 | 4 | 2 | 4 | 4 | 16 |

Tables 1 and 2 show that PHA is effective in inhibiting various F. solani strains.

### Example 2: Antimicrobial Efficacy

Table 3 shows the summary of the zeta potential, antimicrobial (both antifungal and antibacterial) properties and cytotoxicity for mammalian cells of PHA of various chain length.

TABLE 3
Antimicrobial properties & cytotoxicity for mammalian
cells of PHA polymers of various chain length.

| | | | | | | 50% cytotoxic | |
|---|---|---|---|---|---|---|---|
| | | Zeta | | | | concentration (CC₅₀), | |
| | Molecular | potential, | MIC in mg/mL against | | | mg/mL for | |
| Polymer | Weight | mV | S. aureus | P. aeruginosa | F. solani | HaCaT | HDF |
| PHA 10 | 2100 | 29.1 ± 1.0 | 16 | 90.7 ± 35.1 | 4.0 | 107.1 | 68.4 |
| PHA 30 | 6200 | 30.0 ± 0.9 | 12.8 ± 0.7 | 53.3 ± 14.3 | 2.0 | 30.3 | 26.9 |
| PHA 50 | 10000 | 29.8 ± 0.6 | 25.6 ± 4.4 | 58.7 ± 14.3 | 2.5 ± 1 | 29.7 | 20.2 |
| PHA 100 | 21000 | 27.8 ± 1.4 | 44.8 ± 17.5 | 64 ± 0 | 3.5 ± 1 | 32.5 | 28.1 |
| PHA 250 | 52000 | 30.2 ± 1.2 | 76.8 ± 28.6 | 106.7 ± 35.1 | 3.5 ± 1 | 69.7 | 44.4 |
| PHA 400 | 83000 | 33.4 ± 1.0 | 128 | 106.7 ± 35.1 | 3.5 ± 1 | 50.7 | 32.5 |

Based on the above results, it was observed that PHA 10 displayed potent antifungal/antibacterial activity and required a higher concentration for inducing cytotoxicity for mammalian cells when compared to other polymers. Therefore, PHA 10 was chosen for further investigation.

### Example 3: Mechanism of Action

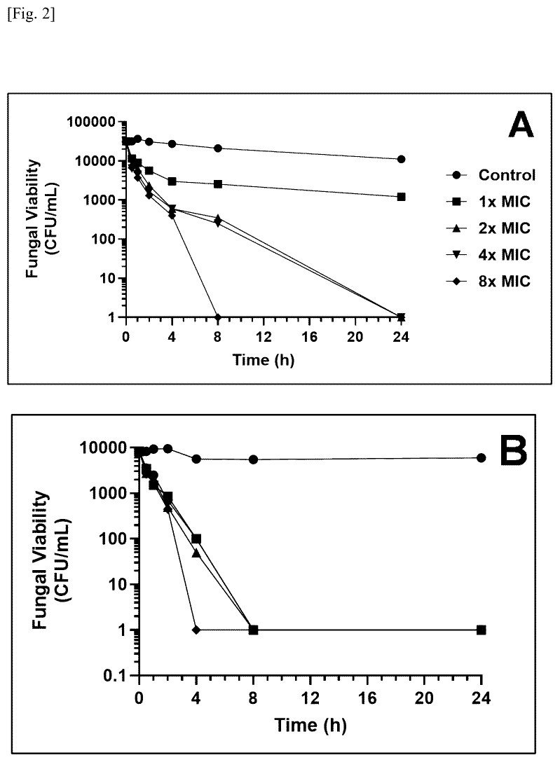

To investigate the mechanism of action of PHA 10, the kill-kinetics of PHA 10 against two F. solani strains (FIGS. 2A and 2B) were firstly investigated. The polymer elicited rapid fungicidal activity against both the strains in a concentration-dependent manner, achieving complete loss of fungal viability between 4 to 8 hours at 8×MIC. The fungicidal properties were both concentration dependent as well as strain dependent, and was shown to be higher than that of natamycin.

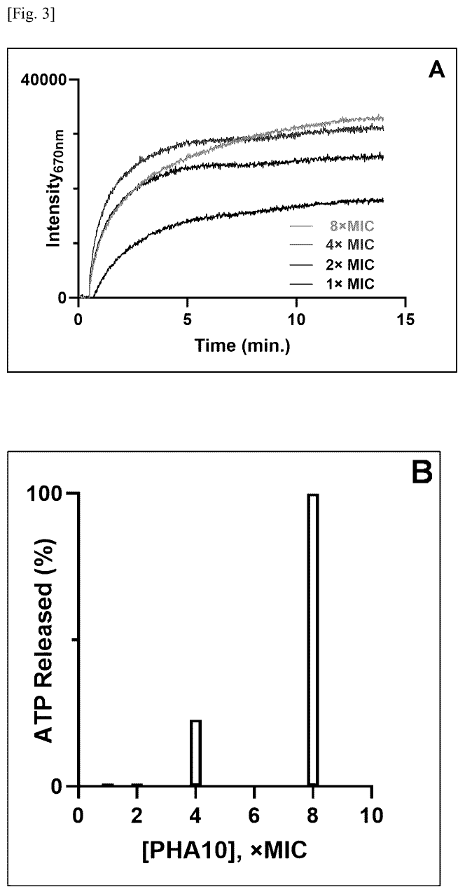

Next, whether the polymer interacts with cytoplasmic membrane of fungi was investigated by monitoring the change in the fluorescence intensity of a membrane potential sensitive probe. As shown in FIG. 3A, the polymer elicited rapid depolarization of the membrane potential with increasing concentration, suggesting membrane disruptive properties.

Further, the release of ATP which is an important intracellular component that is required for fungal survival was determined (FIG. 3B). Exposure for 6 hours at 4× and 8×MIC of PHA 10 resulted in 23% and 100% release of ATP, respectively, whereas no ATP release was observed at 1× and 2×MIC, corroborating the kill-kinetics results. Complete release of ATP was observed at 8×MIC.

### Example 4: Antifungal Activity In Presence of Proteases and Tear Fluid

To mimic the protease rich environments experienced during bacterial or fungal infections, the antifungal activity of PHA 10 was determined in the presence of trypsin, MMP-9 and tear fluid.

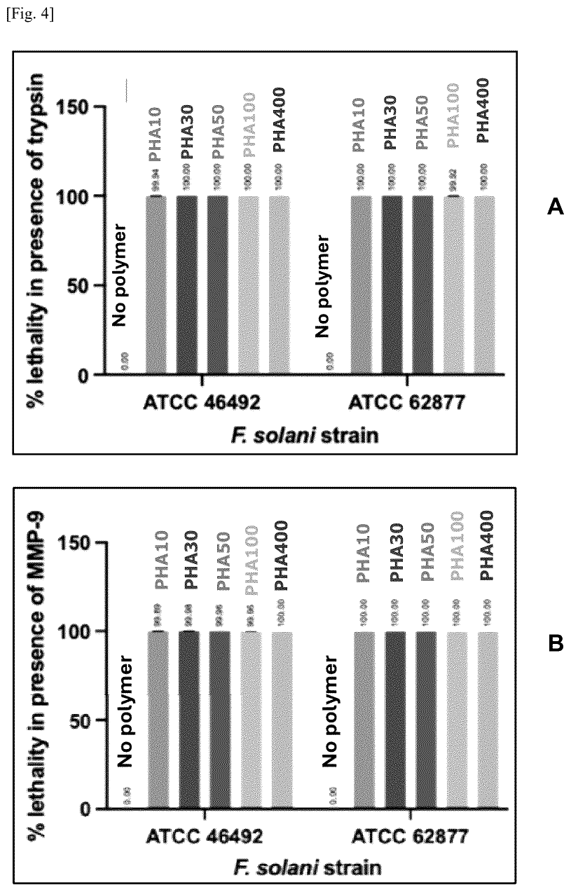

PHA 10 (4×MIC) was incubated with trypsin or MMP-9 (enzyme:PHA 10 ratio=1:100) for 1 hour at 37° C. (pH 7.4) and the mixture was added to the fungal inoculum. Fungal viability was then enumerated after 48 hours incubation. The results are summarised in FIG. 4. No obvious growth was observed in the presence of both the enzymes, indicating that the polymer retained the antifungal activity in the presence of mammalian proteases.

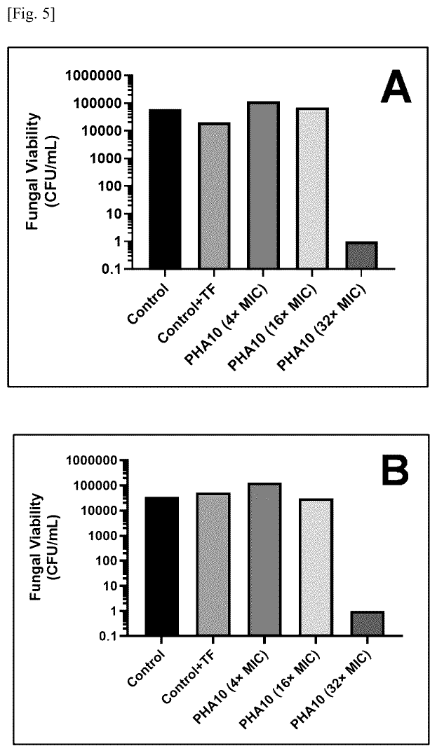

The same investigation was performed in 50% tear fluid to simulate exposure to ocular surface environment. At 4× and 16×MIC of PHA 10 (i.e., 8 and 32 mg/mL) complete loss of activity was observed as indicated by substantial growth of fungi (FIG. 5). When the concentration of polymer was increased to 64 mg/mL (32×MIC), no fungal growth was observed, suggesting that a higher concentration was necessary to elicit fungicidal effect in the presence of 50% tear fluid. These observations suggest that the polymer may bind to some of the tear fluid components thus decreasing the amount of free polymer for fungicidal activity.

### Example 5: Fungal Hyphae Inhibition

The hyphal form of fungi is largely responsible for deep penetration into the corneal tissue and the concomitant tissue destruction. Therefore, the fungal hyphal inhibitory properties of the polymers were determined.

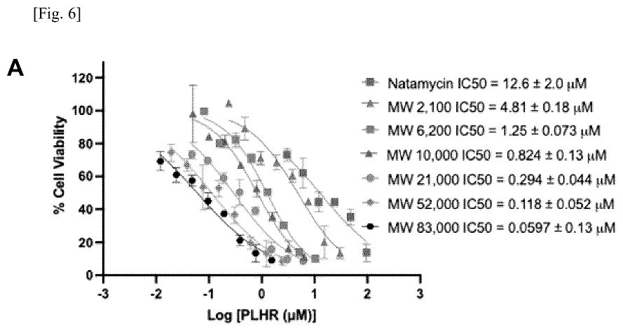

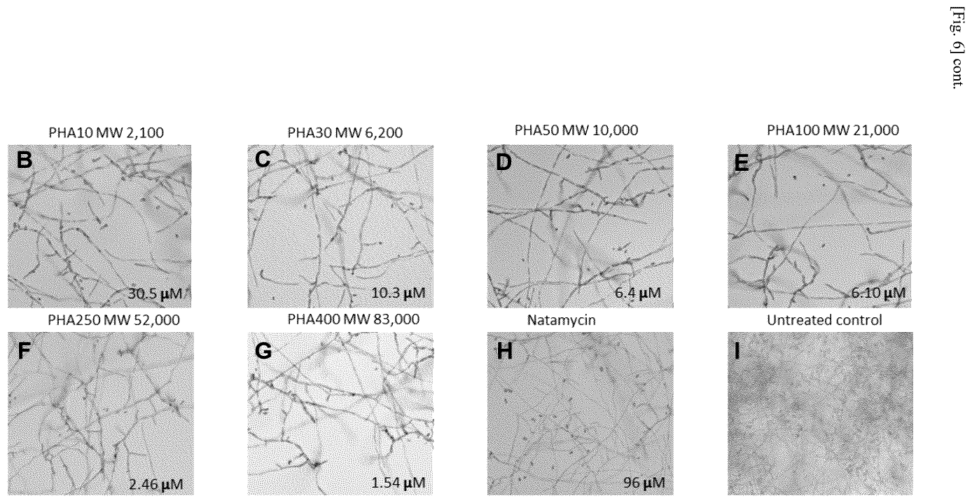

FIG. 6 shows that PHA 10 displayed similar fungal hyphal inhibition as natamycin. However, PHA 10 treatment increased “hyphal breaks”, which indicated that the action of PHA 10 may be different to natamycin, as natamycin did not cause hyphal filaments.

### Example 6: Acute Corneal Toxicity

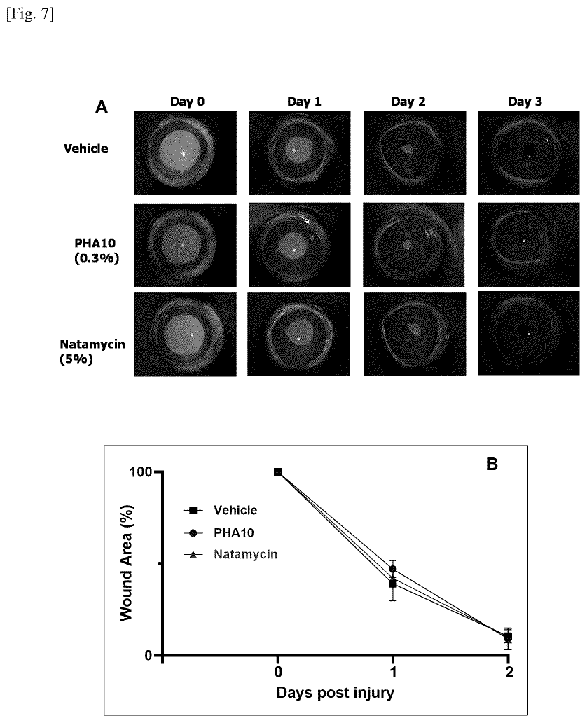

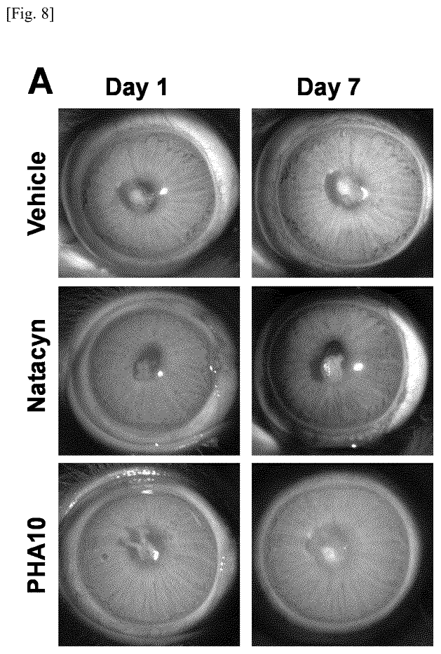

The ocular toxicity of PHA 10 (0.3% w/v in PBS, pH 7.0) and commercial natamycin (5% suspension) formulation were determined in a rabbit model of corneal wound healing. After creating a full-thickness corneal epithelial injury vehicle, PHA 10 or natamycin suspension were added topically at 4 times/day and the progression of wound healing was monitored after fluorescein isothiocynate (FITC) staining and fluorescent slit lamp microscope (FIGS. 7A and 7B). It could be seen that the fluorescein stained area (which appear as the light grey circular spots) began to decrease as the treatment with vehicle, PHA or natamycin continued, suggesting complete wound closure. The results suggest no statistically significant difference between vehicle or treatment groups, indicating that 0.3% PHA 10 was safe for topical instillation to an injured cornea. In addition, no adverse effects such as corneal neovascularization, oedema, hyperemia or conjunctival chemosis was observed, further confirming that the polymer concentration was safe for topical instillation.

### Example 7: Antifungal Efficacy In A Rabit Model

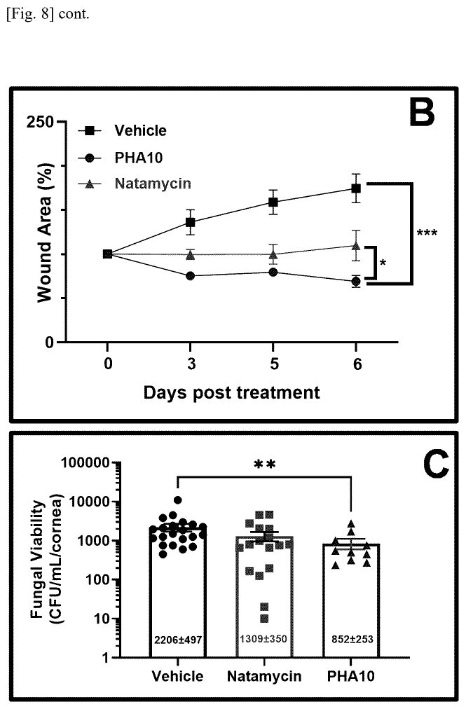

Antifungal efficacy of PHA 10 and 5% natamycin suspension was evaluated in a rabbit model of Fusarium keratitis. The wound area and corneal opacity were lower for the treatment group than untreated control (FIG. 8A). When compared to natamycin, eyes that received PHA 10 had lower wound area than natamycin or untreated control (FIG. 8B). The average fungal bioburden in the PHA 10 treated cornea was significantly lower than untreated control and natamycin, indicating that the polymer decreased the fungal burden in the cornea even compared to natamycin (FIG. 8C).

### Example 8: Antifungal Efficacy In A Mice Model

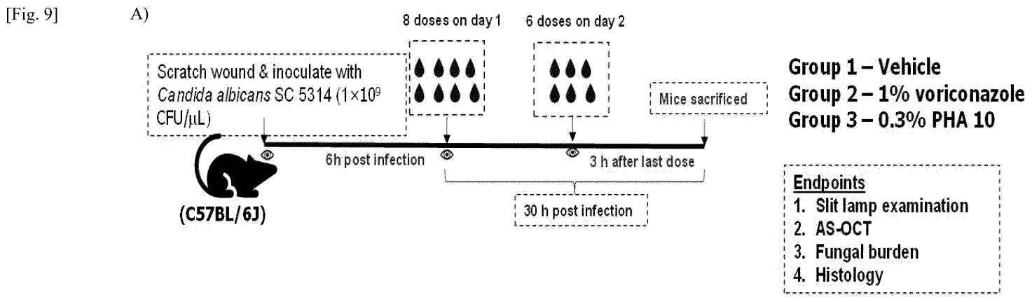

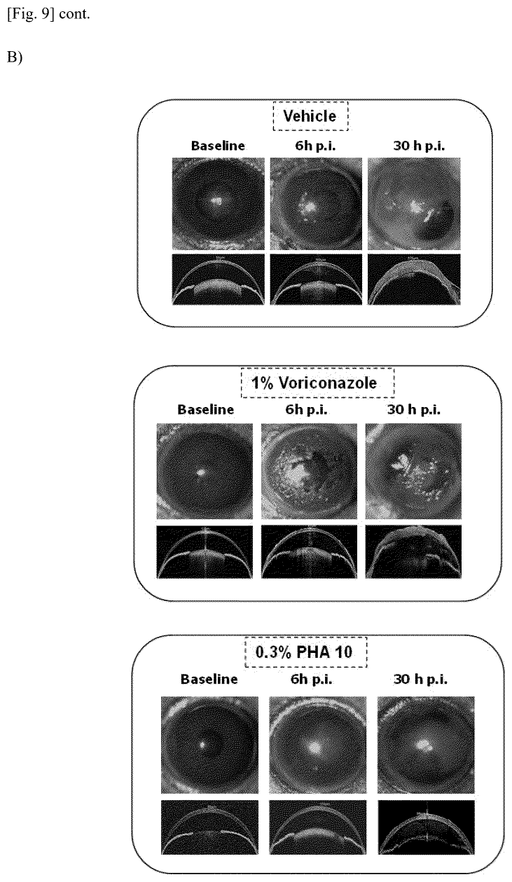

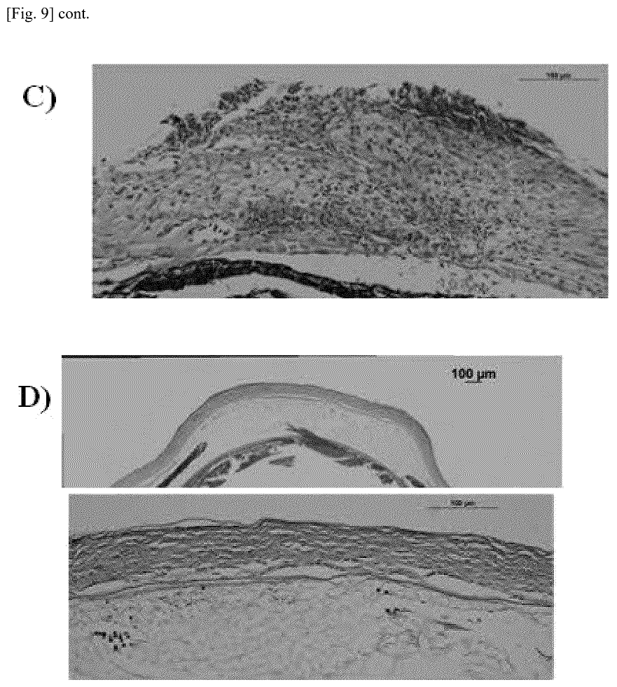

The antifungal efficacy of PHA 10 in a mice model of Candida albicans (SC 5314) keratitis was also tested. Mice infected with C. albicans produced corneal haze at 6 hours post inoculation (p.i.). FIG. 9A depicts the experimental strategies used to assess the antifungal properties of PHA 10 and comparator antifungal. Treatment with vehicle alone resulted in visible neovascularization, corneal haze and ocular surface irregularities at 30 hours p.i. (FIG. 9B). Anterior Segment Optical Coherence Tomography (AS-OCT) images further confirmed the significant presence of hyper reflective materials at 6 hours p.i. that progressed to anterior synechia (fusion of the iris to the cornea) and a decrease in anterior chamber depth, with signs of inflammation that penetrated deep into the lens surface (FIG. 9B). Treatment with PHA 10 was effective in preventing the early inflammatory response and completely abrogated the neovascularization, although mild corneal haze was still present. However, treatment with 1% voriconazole was ineffective as the corneal morphology was similar to that of untreated control, suggesting that the treatment was non-responsive. Hemotoxylin & Eosin (H&E) staining of the excised corneal tissues for the PHA 10 or Voriconazole treated eyes corroborated the AS-OCT results (FIGS. 9C and 9D).

### Example 9: Effect of Molecular Weight On Antifungal Efficacy In A Mice Model

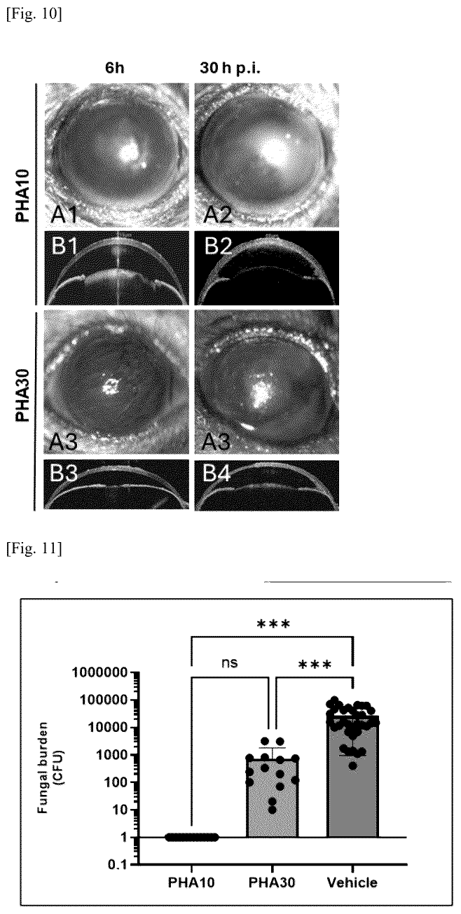

To investigate the effect of molecular weight on in vivo antifungal activity, the efficacy of PHA 10 and PHA30 in a mouse of Candida keratitis was compared. As shown in FIG. 10 and FIG. 11, the results suggest substantial clearance of the pathogens in both the polymer-treated eyes when compared to untreated control.

Slit lamp images and AS-OCT images (FIG. 10) indicated significant changes in the ocular surface after treatment with both polymers, when compared to untreated control or voriconazole treatments. To discern the differences, the fungal burden in the excised cornea after treatment with PHA10 and PHA 30 was determined. No colonies could be detected in the infected eyes that received PHA 10 treatment whereas eyes treated with PHA30 contained 738±1054 (mean±sd) CFU/mL and vehicle treated eyes contained 26362±25414 CFU/mL (FIG. 11). Statistical analysis of the bioburden by one way Turkey's multiple comparison test indicated a significant difference between vehicle and both the polymer treated groups (p. 0.001) whereas no significant difference was observed between PHA10 and PHA30. However, comparison by Mann-Whitney test confirmed significant difference between the groups (p. 0.0001) suggesting significant difference in antifungal activity between the two polymers.

### Example 10: Antifungal Efficacy In An Invasive Candida Keratitis

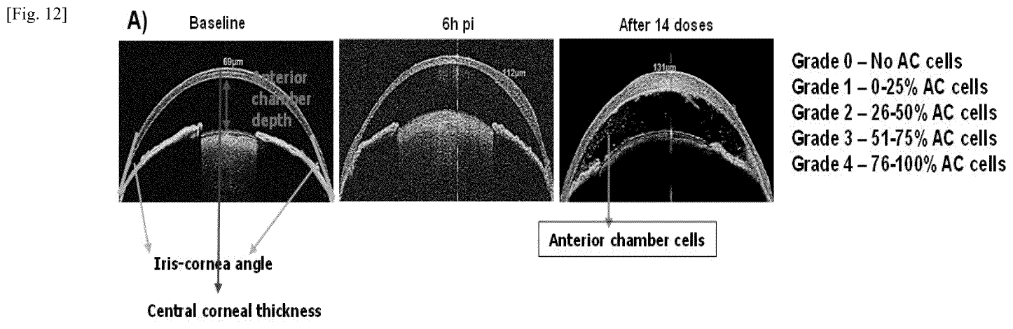

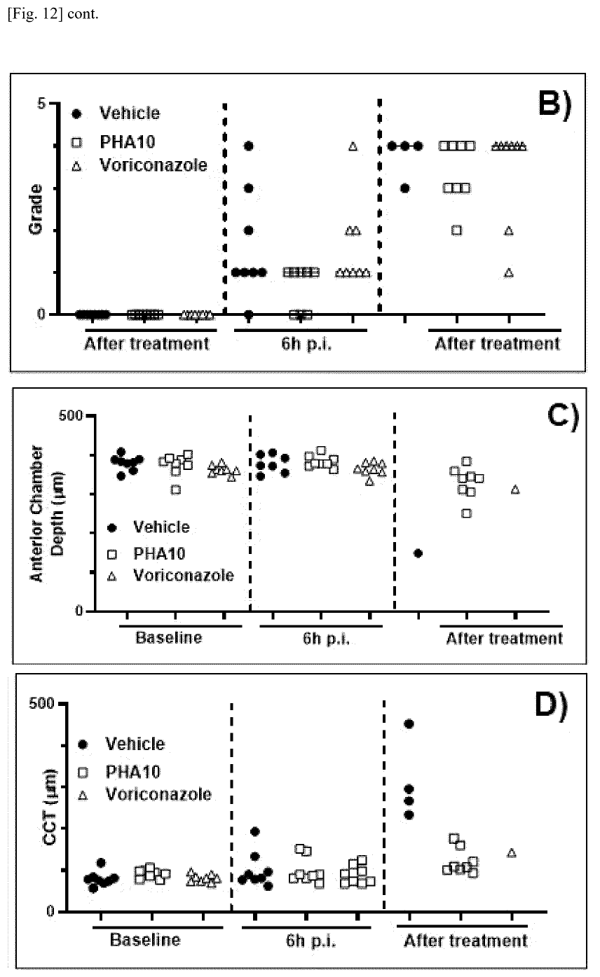

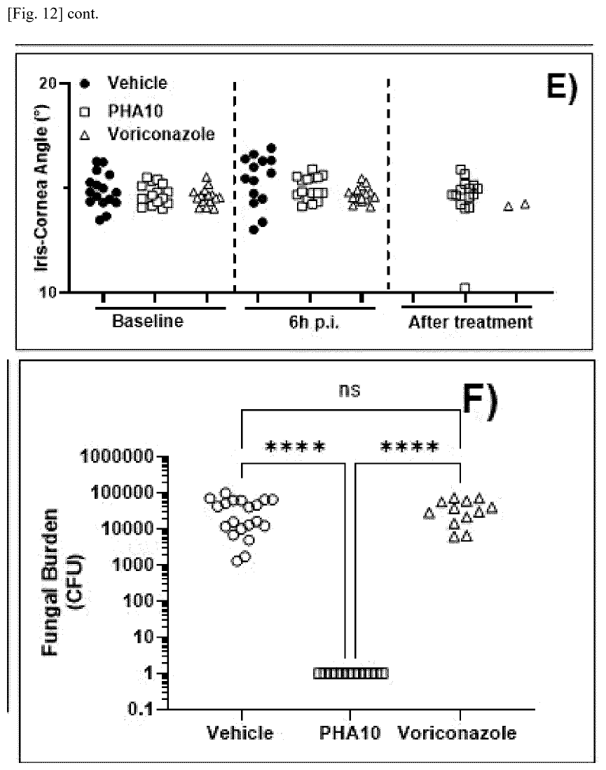

To obtain a better insight into the ability of PHA 10 in attenuating the anterior chamber (AC) inflammation, the AS-OCT results was analysed for all the eyes treated with various groups. FIG. 12A shows the various quantitative descriptive parameters that describe the presence or absence of anterior chamber inflammation. The % AC cells was graded from 0 for the absence of any hyper reflective cells in the AC or 4 that had the greatest prevalence. 4 out of 8 eyes that received PHA 10 treatment had Grade 4 inflammation compared to 6 out of 8 for the voriconazole treated eyes (FIG. 12B). The AC depth remained unchanged for the eyes that received PHA 10 treatment whereas AC depth for only one eye that received voriconazole treatment could be measured due to synechia (FIG. 12C). Similar results were seen for the other two descriptors such as central corneal thickness (CCT) and iris-cornea angle (FIG. 12D and FIG. 12E). Fungal enumeration from the excised eye (n=4) indicated a meant±s.e. of 96400±44200 (range 5000-302000) CFU/mL after 6 hours p.i. (FIG. 12F). The value decreased to 34800±6450 (range 1300-98000) CFU/mL after 24 hours p.i.. Treatment with 1% voriconazole did not decrease the fungal bioburden (34900±6800, range 6200-72800 CFU/mL) at 24 hours p.i., indicating that the azole treatment was non-responsive. However, no colonies were detected for the mice that received 0.3% PHA treatment (n=6 eyes), suggesting that the polymer diminished the fungal survival thereby eradicating bioburden. These observations establish potent antifungal activity of PHA 10 over voriconazole. The above showed that PHA 10 has outstanding anti Candida activities (below detection limit) in all the samples with a superior clinical outcome.

### Example 11: Antibacterial Efficacy in Pseudomonas aeruginosa

Table 4 below shows the antibacterial inhibitory concentration of PHA of various chain length against different strains of Pseudomonas aeruginosa.

TABLE 4
Antibacterial properties of PHA polymers of various chain length.

| | MIC in mg/mL against | | |
|---|---|---|---|
| Polymer | PA 9027 | PA23155 | PA27853 |
| PHA 10 | 32 | 32 | 64 |
| PHA 30 | 4 | 2 | 4 |
| PHA 50 | 2 | 2 | 2 |
| PHA 100 | 4 | 2 | 2 |
| PHA 250 | 4 | 2 | 4 |
| PHA 400 | 4 | 2 | 4 |

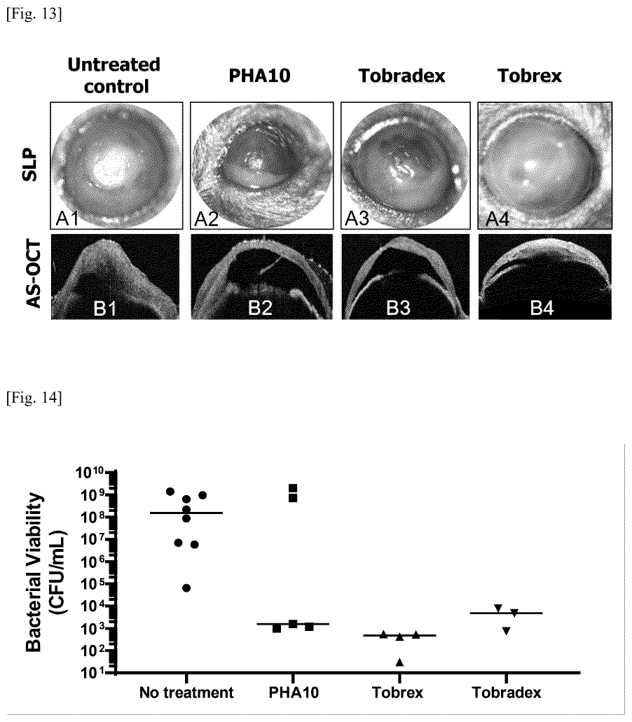

In an animal model, eyes that received PHA10 treatment displayed significant reduction in inflammation and bacterial burden when compared to untreated control and at a level comparable to Tobrex treatment (FIG. 13 and FIG. 14).

### Example 12: Use in Contact Lenses

The PHA 10 may be administered by adsorbing solution comprising PHA 10 into a soft contact lens and having a patient wear the contact lens in the affected eye. The use of contact lenses may enhance the bioavailability of the PHA 10 compared to an eye drop application. This may minimise the frequency of application of the PHA 10, which may in turn increase patient compliance and decrease drug wastage.

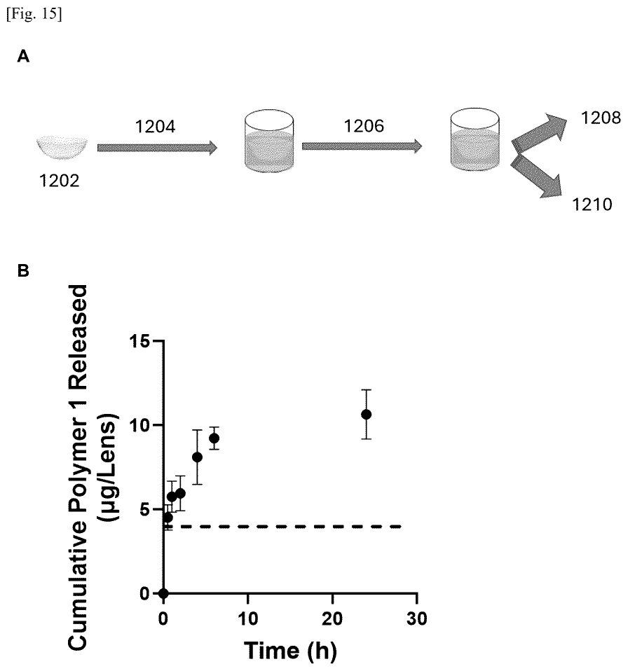

FIG. 15A is a schematic diagram showing how such a contact lens was prepared. An Etafilcon A-3.0D (low myopic) soft contact lens (1202) underwent preparation (1204) comprising the following steps:
1) The lens was soaked in PBS (pH 7.4) for 1 hour;
2) The lens was blotted onto lint free paper; and
3) The lens was transferred to an amber glass vial containing 0.01% (w/v) PHA 10 for 24 hours.

Subsequently, preparation (1206) was applied comprising the following steps:
4) Autoclaved at 110° C. for 18 minutes; and
5) Equilibrated at room temperature for 48 hours.

The contact lens was then analysed for polymer content (1208) and polymer release kinetics (1210).

FIG. 15B shows the cumulative release profile of the PHA 10 from the contact lens. It was shown that PHA 10 remained stable under autoclave conditions. FIG. 15B also shows that the MIC of PHA 10 was maintained for at least 20 hours.

With high fungal membrane selectivity, outstanding toxicity profile and efficacy profile in in vivo experimental models, PHA 10 may be useful in treating filamentous and non-filamentous fungal keratitis.

### INDUSTRIAL APPLICABILITY

Polyhomoarginine comprising 6-26 homoarginine residues or a pharmaceutically acceptable salt thereof may be useful in treating an infection, in particular a fungal infection, a bacterial infection or a combination thereof. The present invention may also be useful in preparing a formulation for administering the polyhomoarginine comprising 6-26 homoarginine residues or a pharmaceutically acceptable salt thereof to a patient, or in preparing a contact lens comprising polyhomoarginine comprising 6-26 homoarginine residues or a pharmaceutically acceptable salt thereof.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1-20. (canceled)

21. A method of treating an infection, wherein the infection is a fungal infection, a bacterial infection or a combination thereof, comprising administering a polyhomoarginine comprising 6-26 homoarginine residues or a pharmaceutically acceptable salt thereof to a subject.

22. The method according to claim 21, wherein the polyhomoarginine comprises 8-12 homoarginine residues, preferably 10 homoarginine residues, or wherein the polyhomoarginine has an average molecular weight in the range of about 2,000 to about 6,000 Da.

23. The method according to claim 21, wherein the polyhomoarginine comprises L-homoarginine and/or D-homoarginine, or wherein the polyhomoarginine is optically pure, or wherein the polyhomoarginine consists only of L-homoarginine.

24. The method according to claim 21, wherein the fungal infection is caused by a fungus selected from the group consisting of Candida species, Fusarium species, Aspergillus species, Cryptococcus species and any mixture thereof, or the fungal infection is caused by a fungus selected from the group consisting of C. albicans, C. parapsilosis, C. tropicalis, C. guillermondii, C. krusei, F. solani, F. dimerum, F. oxysporum, F. sacchari, and F. verticillioides, F. polyphialidicum, A. flavus, A. effusus, A. tamarii, A. sydowii, A. protuberus and A. terreus and any mixture thereof.

25. The method according to claim 21, wherein the bacterial infection is caused by gram-positive bacteria and/or gram-negative bacteria, or the bacterial infection is caused by a bacteria selected from the group consisting of Staphylococcus species, Streptococcus species, Pseudomonas species, Enterobacteriaceae, and any mixture thereof, or the bacterial infection is caused by a bacteria selected from the group consisting of S. aureus, Methicillin-resistant S. aureus, S. epidermidis, S. pneumoniae, P. aeruginosa, and any mixture thereof.

26. The method according to claim 21, wherein the infection further comprises an infection caused by an amoeba, preferably Acanthamoeba species.

27. The method according to claim 21, wherein the infection is an infection of the skin, or the infection is an infection of the eye, or the infection is an infection of the cornea of the eye, or the infection is keratitis.

28. The method according to claim 21, wherein the polyhomoarginine is to be administered topically, intrastromally, intracamerally, or by subconjunctival injection.

29. The method according to claim 21, wherein the polyhomoarginine is to be administered in a formulation having a concentration in the range of 50 μg/mL to 50 mg/mL, or the polyhomoarginine is to be administered 1 to 12 times per day.

30. The method according to claim 21, comprising a further antimicrobial agent, preferably an antibacterial agent or an antifungal agent, preferably wherein the further antimicrobial agent is selected from the group consisting of amphotericin B, natamycin, fluconazole, ketaconazole, voriconazole, miconazole, luliconazole and any mixture thereof.

31. The method according to claim 30, wherein the further antimicrobial agent is selected from the group consisting of amphotericin B, natamycin, fluconazole, ketaconazole, voriconazole, miconazole, luliconazole and any mixture thereof.

32. A contact lens comprising polyhomoarginine comprising 6-26 homoarginine residues or a pharmaceutically acceptable salt thereof.

33. A method of treating an infection, wherein the infection is a fungal infection, a bacterial infection or a combination thereof, comprising applying the contact lens of claim 32 to an eye of a subject.
